(19) United States
(12) Patent Application Publication
(10) Pub. No.: US 2004/0147883 A1
(43) Pub. Date: Jul. 29, 2004

(54) Liquid aspirating and medicine adding injection needle

(76) Inventor: Ming-Hsiung Tsai, Taichung (TW)

Correspondence Address: Ming-Hsiung Tsai, P.O. Box 2103, Taichung, TW

(21) Appl. No.: 10/351,987

(22) Filed: Jan. 28, 2003

(51) Int. Cl. A61M 5/32

(52) U.S. Cl. 604/274

(57) ABSTRACT

A liquid aspirating and medicine adding injection needle includes a needle body with only about 1 cm length, a needle barrel and a needle head respectively positioned at two ends of the needle body. The injection needle is formed with a perforation passing through the needle body from outer side to inner side. The needle head has a diameter tapered to the front end and has a conic pattern. The front end of the needle head is obtuse so as to avoid impalement of human body.

### BACKGROUND OF THE INVENTION

The present invention is related to a liquid aspirating and medicine adding injection needle, and more particularly to a liquid aspirating and medicine adding injection needle having an obtuse needle head so as to avoid a sudden impalement of human body. Needle impalement is a significant risk in health care profession industry. The needle impalement may result in B-type hepatitis, C-type hepatitis, AIDS, etc. According to statistic data from Council of Labor Affairs Taiwan in 1997, 74.8% professionals may be impaled or cut by needle or sharp piece. Among these workers, medical professionals are potentially in most danger of needle impalement. Relevant papers indicate that the needle impalement mostly takes place when a worker fit the needle sheath back onto the needle. This is indirectly related to the 3.8 cm length of the existenting 18 to 22 gauge needle.

FIG. 8 shows a syringe having an injection needle 8 with very sharp tip 81. The sharp tip 81 can be easily thrust into human skin. However, a medical worker could be easily impaled by the tip 81.

The container of drug agents has two types, an ampule with an all glass bottle and a vial with a rubber-sealed glass bottle type. The neck of the ampule is cut down by a diamond knife and then allow an injection needle to be extended into the bottle to aspirate the liquid medicine. With respect to vial , the tip of the injection needle is thrust through the rubber film sealer the opening of the bottle and then the injection needle is extended into the bottle for aspirating liquid medicine. The injection needle can be used to inject the liquid medicine into an infusion set through a medicine adding port at variable sites.

To establish an infusion line, it is necessary to use a catheter needle inserted into the vein of a patient. After the liquid medicine is aspirated out from the glass bottle, the liquid medicine is added into the infusion set from the rubber medicine adding ports of a gravity bottle, a small drug reserving bag or a drug adding tube on the infusion line. In all the above procedures, it is necessary to use the tip 81. (The 18 to 24 gauge needles are mostly common used.) The needle impalement to the professional is most likely to happen in these situations. It is tested and found by this applicant that the rubber film can be penetrated by any hard thrust with a blunt needle. Therefore, it is unnecessary to use the needle with very sharp tip to thrust the rubber film. This is also true when adding the liquid medicine into the medicine adding port of the infusion set which is also sealed by rubber film.

In addition, in paramedical departments such as preparatory room of radiology, dispensing special anticancer drugs of pharmacy and clinical laboratory for bacteria culture, a great amount of injection needles with sharp tips 81 are still frequently used. As a result, needle impalement is quite easy to take place, besides, the cleaning workers are also likely to get impaled by the needles.

### SUMMARY OF THE INVENTION

It is therefore the primary object of the present invention to provide a liquid aspirating and medicine adding injection needle without sharp tip so as to minimize possible impalement of medical professionals and relevant workers.

According to the above object, the liquid aspirating and medicine adding injection needle includes a needle body of only about 1 to 2 cm length, a needle barrel and a needle head positioned at two ends of the needle body respectively. The needle body is tubular and has a hollow interior. One end of the needle head distal from the needle holder is defined as a front end. The needle head has a diameter tapered to the front end and has a conic pattern. The front end is obtuse. The aspirating needle has at least one perforation passing through the needle body from outer side to the hollow interior.

The present invention can be best understood through the following description and accompanying drawings wherein:

### BRIEF DESCRIPTION OF THE DRAWINGS

FIG. 1 is a view showing that the aspirating needle of the present invention is mounted on a barrel;

FIG. 2 is a perspective view of the present invention;

FIG. 3 is an enlarged view of the needle body and needle head of the present invention;

FIG. 4 is a sectional view showing the use of the present invention;

FIG. 5 is a perspective view of a second embodiment of the present invention;

FIG. 6 is an enlarged view of the needle head of the second embodiment of the present invention;

FIG. 7 is a sectional view showing the use of the second embodiment of the present invention; and

FIG. 8 is a perspective view of a conventional injection needle.

### DETAILED DESCRIPTION OF THE PREFERRED EMBODIMENTS

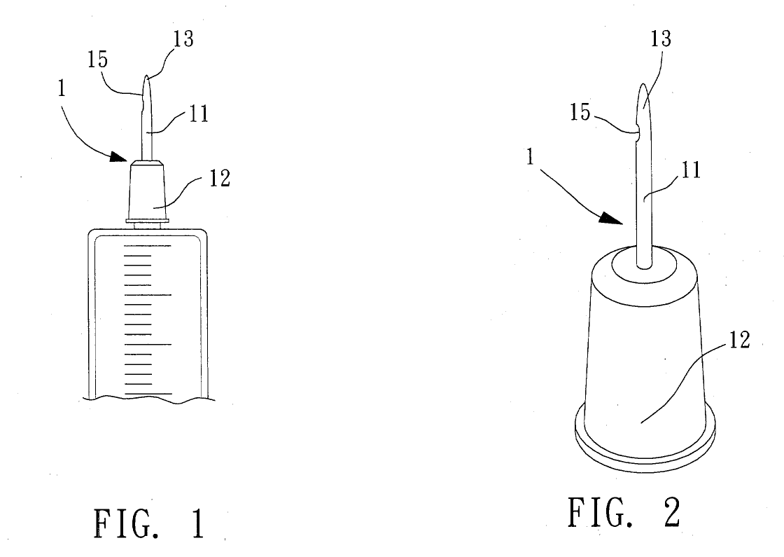

Please refer to FIGS. 1 to 3. The liquid aspirating and medicine adding injection needle 1 of the present invention includes a needle body 11, a needle holder 12 and a needle head 13 respectively positioned at two ends of the needle body 11. The needle body 11 is tubular and has a hollow interior 14. In this embodiment, the needle body 11 has such a specification that the length is about 1 to 2 cm and the external diameter is about 0.8 to 1.2 mm. One end of the needle head 13 distal from the needle holder 12 is defined as a front end 131. The needle head 13 has a diameter tapered to the front end 131 and has a conic pattern. The front end 131 is obtuse. The aspirating needle 1 has a perforation 15 formed on a lateral side of the needle head 13 and passing through the needle body 11 from outer side to the hollow interior 14.

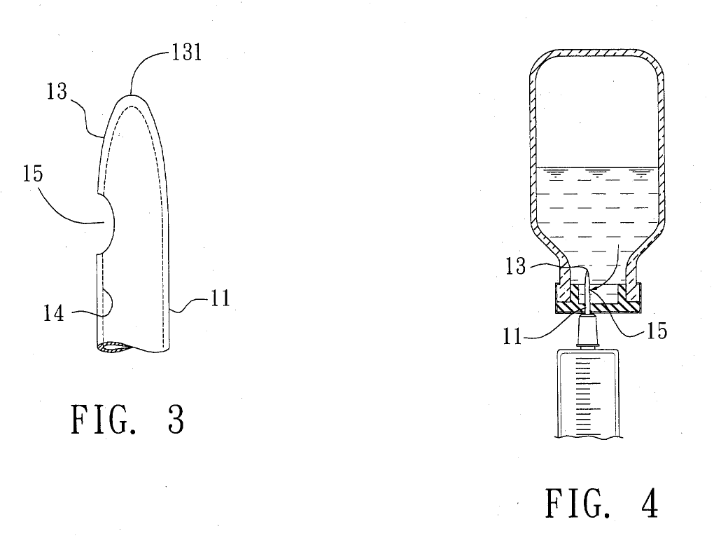

As shown in FIGS. 2 and 3, the front end 131 of the needle head 13 is obtuse so as to avoid impalement of medical professionals. The needle head 13 is conic and the diameter of the obtuse front end 131 is quite small. The needle body 11 is made of stainless steel or special plastic material and has a considerable hardness. Therefore, the front end 131 of the needle head 13 can be slightly forced to thrust through the rubber film sealer the opening of the phial as shown in FIG. 4. The needle body 11 can be then extended into the phial to aspirate the liquid medicine into the perforation 15 of the needle head 13. Similarly, the front end 131 of the needle head 13 can be used to thrust the rubber film of a medicine-adding bottle to add the medicine into the infusion set.

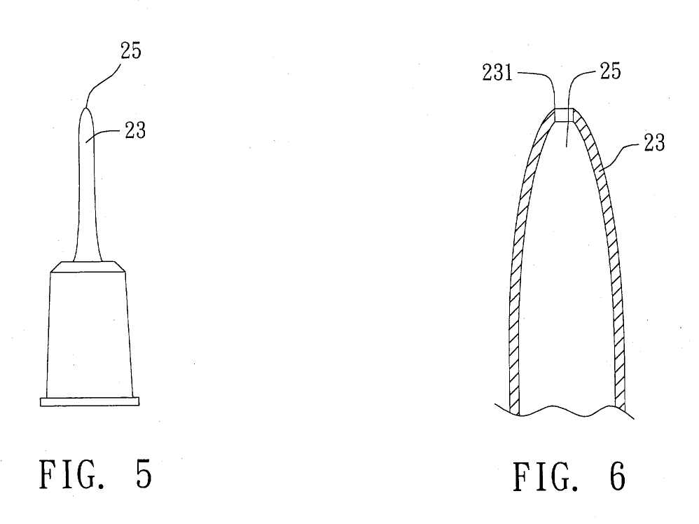

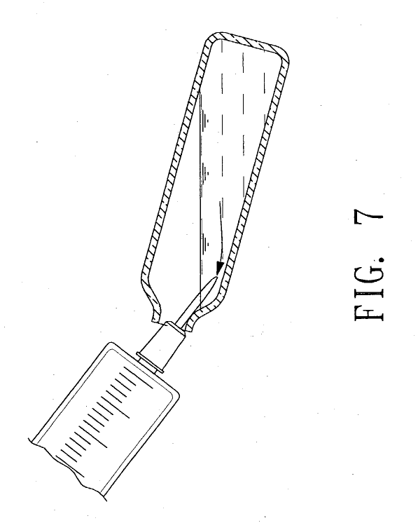

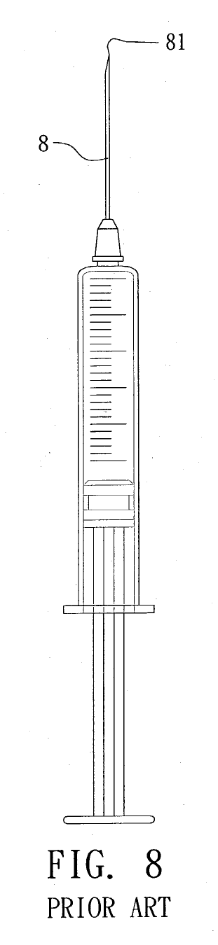

FIGS. 5 and 6 show a second embodiment of the present invention, in which the perforation 25 is formed at front end 231 of the needle head 23 and axially passes through the needle head 23. In such structure, the front end 231 of the needle head 23 is also obtuse to achieve the same effect as the first embodiment. During use, the liquid medicine is aspirated through the perforation 25 at front end 231 of the needle head 23 or injected into the infusion set through the perforation 25 as shown in FIG. 7.

The above embodiments are only used to illustrate the present invention, not intended to limit the scope thereof. Many modifications of the above embodiments can be made without departing from the spirit of the present invention.

What is claimed is:

1. A liquid aspirating and medicine adding injection needle comprising a needle body, a needle barrel and a needle head respectively positioned at two ends of the needle body, the needle body being tubular and having a hollow interior, one end of the needle head distal from the needle barrel being defined as a front end, the needle head having a diameter tapered to the front end and having a conic pattern, the front end being obtuse, the aspirating needle having at least one perforation passing through the needle body from outer side to the hollow interior.

2. The liquid aspirating and medicine adding injection needle as claimed in claim 1, wherein the perforation is formed on a lateral side of the needle head and radially passes through the needle head.

3. The liquid aspirating and medicine adding injection needle as claimed in claim 1, wherein the perforation is formed at front end of the needle head and axially passes through the needle head.

4. The liquid aspirating and medicine adding injection needle as claimed in claim 1, wherein the needle body has such a specification that the length is about 1 to 2 cm and the external diameter is about 0.8 to 1.2 mm.

5. The liquid aspirating and medicine adding injection needle as claimed in claim 1, wherein the needle body is made of metal or plastic material.
